(19) United States
(12) Patent Application Publication
(10) Pub. No.: US 2024/0181113 A1
(43) Pub. Date: Jun. 6, 2024

(54) A GERMICIDAL LIGHT EMITTING DEVICE

(71) Applicant: SIGNIFY HOLDING B.V., EINDHOVEN (NL)

(72) Inventor: TIES VAN BOMMEL, Horst (NL)

(21) Appl. No.: 18/285,247

(22) PCT Filed: Mar. 29, 2022
(86) PCT No.: PCT/EP2022/058211

§ 371 Date: Sep. 30, 2023

(30) Foreign Application Priority Data
Apr. 1, 2021 (EP) 21166689.6

(51) Int. Cl. A61L 9/20

(52) U.S. Cl. CPC A61L 9/20; A61L 2209/11; A61L 2209/12; A61L 2209/15

(57) ABSTRACT

A germicidal light emitting device (1) comprising a housing (10) forming an air inlet (11) and an air outlet (12), a fan (13) configured to provide an air flow (20) through the air inlet (12), from the air inlet (12) to the air outlet (11) and through the air outlet (11), the air flow (20) being directed towards a first direction (D1) and away from a second direction (D2), at least one first UV light source (14) arranged and configured to emit, when in operation, first UV light with a first spectral distribution and a main emission direction being the first direction (D1), at least one second UV light source (15) arranged and configured to emit, when in operation, second UV light with a second spectral distribution and a main emission direction being the second direction (D2), and the first spectral distribution is different from the second spectral distribution.

### FIELD OF THE INVENTION

The invention concerns a germicidal light emitting device comprising a housing forming an air inlet and an air outlet. A fan is provided to facilitate an air flow through the air inlet, from the air inlet to the air outlet and through the air outlet. The air flow is directed away from a first direction and towards a second direction. The germicidal light emitting device is configured to emit UV light onto the air flow.

### BACKGROUND OF THE INVENTION

UV (Ultra-Violet) disinfection has become a topic of renewed interest as the demand for sterilization increases. In general, an increase has been observed in the desire to protect oneself and others from the spread of bacteria and viruses such as influenza or against the outbreak of novel viruses like the recent COVID-19 virus. Currently, the use of UV light is being considered for providing a germicidal effect within living spaces.

However, the germicidal effect of UV light varies within the spectrum of UV light. Furthermore, different bacteria and viruses may be vulnerable to different wavelengths of UV light.

To accommodate for different bacteria and viruses being vulnerable to different wavelengths, germicidal light emitting devices capable of emitting UV-light with different wavelengths has been developed. For instance, TW M585883 discloses an air cleaning device comprising an airflow generating member and a germicidal lamp group. The air generating member is configured for providing an air flow through the air cleaning device. The germicidal lamp group is configured for emitting at least two different wavelengths of germicidal rays onto the airflow.

However, there is still a need for an improved germicidal light emitting device in terms of any one or more of safety, such as to meet demands set in UV dose legislation, and performance.

### SUMMARY OF THE INVENTION

It is an object of the present invention to provide a germicidal light emitting device which is improved in terms of at least one of safety and performance.

These and other objects of the invention are achieved by a germicidal light emitting device comprising: a housing forming an air inlet and an air outlet, a fan configured to provide an air flow through the air inlet, from the air inlet to the air outlet and through the air outlet, the air flow being directed towards a first direction and away from a second direction, at least one first UV light source arranged and configured to emit, when in operation, first UV light with a first spectral distribution and an emission targeted in the first direction along or towards a ceiling/or a wall, at least one second UV light source arranged and configured to emit, when in operation, second UV light with a second spectral distribution and an emission targeted in the second direction, and at least one mounting element configured to enable mounting the germicidal light emitting device on the ceiling or the wall in such a way that the first direction extends in parallel to the ceiling or the wall, wherein the first UV light is UV-C and the second UV light is at least one of UV-A and/or UV-B, and wherein at least one of the first and second UV light source are arranged and configured for emitting light out of the housing.

By having different spectral distributions in different directions, it allows for a germicidal light emitting device which is modifiable to different situations. For example, the UV light in the first direction may be configured to have a germicidal effect towards a first group of bacteria and viruses, while the UV light in the second direction may be configured to have a germicidal effect towards a second group of bacteria and viruses. Furthermore, by having the UV-light targeted in different directions, the first UV light and the second UV light may be emitted towards different critical areas where the first UV light and the second UV light may have the highest effect. Said germicidal killing effect is further improved by a relatively long exposure time of the air flow to the UV-light, yet without the germicidal light emitting device becoming relatively spacious/bulky, because the at least one of the first and second UV light source are arranged and configured for emitting light out of the housing (when in operation). Thereto, preferably the germicidal light emitting device may have the feature that both first and second UV light source are arranged and configured for emitting light out of the housing. Furthermore, by drawing surrounding air towards the germicidal light emitting device, via the fan, it may facilitate an efficient and fast sterilization of the air in the surroundings of the germicidal light emitting device. Furthermore, as the different light sources are configured emit light towards different areas interference between the emitted light is minimized. Consequently, a germicidal light emitting device is provided which is improved in terms of both safety, particularly by allowing effective disinfection by relatively low doses of UV light, and performance, particularly by allowing for irradiating different bacteria and viruses being vulnerable to different wavelengths.

In the context of the invention a main emission direction may be interpreted as the direction away from a light source with the highest intensity of light emitted from the light source. The main emission direction may alternatively be interpreted as the average direction of light emitted away from the light source, e.g. the main emission direction may be obtained by averaging the directions in which the light source emits light.

In the context of the invention a spectral distribution may be interpreted as a group of wavelengths, or just a single wavelength.

The housing may be an opaque housing or a transparent housing.

The fan may be arranged within the housing, at the air inlet or at the air outlet. The fan is configured for facilitating an air flow through the air inlet and the air outlet.

In an embodiment, the germicidal light emitting device comprises a plurality of first UV light sources arranged and configured to emit, when in operation, first UV light with a first spectral distribution and a main emission direction being the first direction. In an additional or alternative embodiment, the germicidal light emitting device further comprises a plurality of second UV light sources arranged and configured to emit, when in operation, second UV light with a second spectral distribution and a main emission direction being the second direction.

Thereby, a light emitting device is provided which allows for one or more of a light output with a higher intensity and a wider spectral distribution. Thus, the light emitting device is improved even further in terms of both safety and performance. To further improve the safety of the germicidal light emitting device, it may have the feature that both first and second UV light are targeted along or towards the ceiling and/or a wall, i.e. in directions away from people.

In an embodiment, the air inlet is configured to collimate the first UV light along the first direction, and the air outlet is configured to collimate the second UV light along the second direction.

Consequently, UV light emitted from the germicidal light emitting device may be directed as desired towards critical areas. Furthermore, by reducing the spread of the UV light emitted a higher intensity exposure at certain areas is obtained. The air inlet and air outlet may further be provided with shapes which facilitates the collimation of light, e.g. slits or pin holes.

In an embodiment, the germicidal light emitting device further comprises a first collimator and a second collimator, wherein the first collimator is arranged at the first UV light source and configured to collimate the first UV light along the first direction, and wherein the second collimator is arranged at the second UV light source and configured to collimate the second UV light along the second direction.

By providing a collimator for collimating light it allows for the shape of the air inlet and the air outlet to be chosen without having to consider the need for collimating light. The first collimator and the second collimator may be an integral part of the first UV light source and the second UV light source, respectively. Alternatively, the first collimator and the second collimator may be provided as external components which are arrangeable at the first UV light source and the second UV light source, respectively. The first collimator and the second collimator may be provided as lenses, mirrors, or other optical components.

In an embodiment, the first direction is non-parallel with the second direction. In an embodiment, the first direction is perpendicular to the second direction. In an embodiment, the first direction is opposite the second direction.

In an embodiment, the first UV light is UV-C light and the second UV light is one or more of UV-B and UV-A light.

Consequently, the first spectral distribution may comprise one or more wavelengths within the UV-C spectrum, 100 nm-280 nm, and the second spectral distribution may comprise one or more wavelengths within the UV-A spectrum, 320 nm-400 nm, and/or one or more wavelengths with the UV-B spectrum, 280 nm-320 nm. As said, it is especially advantageous, that the germicidal light emitting device is placed in a space where people are present, that potentially harmful UV-C light is targeted in a first direction away from where people go about, e.g. towards a wall or a ceiling or close to and parallel with a ceiling, and that only the UV-A and/or UV-B may be targeted in a direction where people are present. Consequently, the strong germicidal effect from the UV-C light is still achieved, without posing a risk to any people. However, recent studies have shown that deep UV-C light, 200 nm-230 nm, may not be harmful to people, as a result of a low skin penetration depth, such deep UV-C light may in some, not claimed, embodiments be targeted to where people are present.

In an embodiment, the UV-C light is far-UV light which is UV-C light in the wavelength range from 190-230 nm. Far-UV is relatively less unsafe compared to higher UV-C wavelengths. Also, the disinfection performance of the wavelength range of 190-230 nm is higher compared to higher UV-C wavelengths.

In an embodiment, the UV-A light is in the wavelength range from 340-380 nm. Light this wavelength range is relatively safer than lower UV-A wavelength range whereas it still provides good disinfection in terms of killing bacteria.

In an embodiment, the first UV light comprises a first spatial light distribution having a first full-width-half-maximum, FWHM1 and the second UV light comprises a second spatial light distribution having a second full-width-half-maximum, FWHM2, wherein FWHM1<FWHM2, preferably FWHM1<0.5*FWHM2, more preferably FWHM1<0.3*FWHM2 and most preferably FWHM1<0.1*FWHM2.

Thereby, the first UV light may be used for upper air disinfection, while the second UV light may be used full air disinfection. Especially, if the first UV light is less safe (or unsafe) with respect to the second UV light, then there are more constrains (e.g. due to legislation) and the first UV light should have a higher degree of collimation than the second UV light.

In an embodiment, the FWHM1<3 degrees, the FWHM1<2 degrees or the FWHM1<1.5 degrees. A light absorbing collimator, e.g. light absorbing slits, may be used to create this narrow distribution. Also, the FWHM2 may be created with a different collimator or collimator arrangement than the FWHM1.

In an embodiment, the germicidal light emitting device further comprises a third UV light source configured to emit, when in operation, third UV light with a third spectral distribution and a main emission direction being the third direction, wherein the third spectral distribution is different from the first spectral distribution and the second spectral distribution.

The third UV light may be emitted in the first direction. Alternatively, or in addition, the third UV light may be emitted in the second direction. The third light may particularly be emitted in a third direction different from the first and second direction. The germicidal light emitting device may comprise one or more of a second air inlet and a second air outlet. The third UV light may be aligned with the second air outlet or the second air inlet.

Consequently, the air flow passing through the germicidal light emitting device may be sterilized by three different UV lights. For example, the first UV light may be UV-C light, the second UV light may be UV-A light, and the third UV light may be UV-B light. Furthermore, as the different light sources are configured emit light towards different areas interference between the UV light is not needed to be considered. The third UV light source may be arranged to radiate light at the air flow passing through the fan.

In an additional embodiment, where the germicidal light emitting device further comprises a third UV light source configured to emit, when in operation, third UV light with a third spectral distribution and a main emission direction being the third direction, where the third spectral distribution is different from the first spectral distribution and the second spectral distribution, the third direction may be different from the first and second direction. In another additional embodiment, one or more of a second air inlet and a second air outlet may be provided, and one of these may be aligned with the emission direction of the third UV light.

In an embodiment, the germicidal light emitting device further comprises at least one mounting element configured to enable mounting the germicidal light emitting device on a surface. The surface may e.g. be a wall or a ceiling.

Consequently, the germicidal light emitting device may be arranged at almost any desired location. The mounting element may be a mounting bracket or a light fixture.

In an embodiment the air inlet and/or the air outlet comprises a plurality of slits, holes, or openings.

Consequently, the air flow through the germicidal light emitting device is not obstructed, while still allowing for light passing through the air inlet and/or the air outlet to be collimated.

In an embodiment the first UV light source and/or the second UV light source are arranged inside the housing.

Consequently, the components of the germicidal light emitting device are protected by the housing. Furthermore, by arranging a light source within the housing it may be assured that light leaving through the air inlet and/or the air outlet is collimated by the air inlet and/or the air outlet. It may also be assured that light is leaving the housing only through the inlet and outlet such as to obtain an improved directional control of the UV light.

Alternatively, the first UV light source and/or the second UV light source are arranged on an exterior of the housing. This may ease the accessibility of the first UV light source and/or the second UV light source, thus facilitating adjustment and/or exchange of the first UV light source and/or the second UV light source.

In an embodiment, at least one of the first UV light source and the second UV light source is arranged outside of the housing.

In an embodiment, the germicidal light emitting device further comprises a controller for individually controlling one or more of the first UV light source, the second UV light source and, where provided, the third UV light source.

The objects of the invention are further achieved by a luminaire or a lamp comprising a germicidal light emitting device according to the invention and a light source emitting, when in operation, white light.

The white light may be emitted in one or more of the first and the second direction. Most often, however, the white light is emitted in the second direction, e.g. towards the floor. The white light may have a color temperature in the range from 2000 to 6000 K.

The white light may have a CRI of at least 80.

In an embodiment, the luminaire or a lamp comprises a germicidal light emitting device, where the first UV light is UV-C light and the second UV light is one or more of UV-B and UV-A light, where the first UV light comprises a first spatial light distribution having a first full-width-half-maximum, FWHM1, and the second UV light comprises a second spatial light distribution having a second full-width-half-maximum, FWHM2, where FWHM1<FWHM2, and where the white light has a main emission direction being the second direction.

It is noted that the invention relates to all possible combinations of features recited in the claims. Other objectives, features, and advantages of the present inventive concept will appear from the following detailed disclosure, from the attached claims as well as from the drawings. A feature described in relation to one of the aspects may also be incorporated in the other aspect, and the advantage of the feature is applicable to all aspects in which it is incorporated.

### BRIEF DESCRIPTION OF THE DRAWINGS

This and other aspects of the present invention will now be described in more detail, with reference to the appended drawings showing embodiments of the invention.

FIG. 1 shows a schematic cross-sectional view of a germicidal light emitting device according to an embodiment of the invention.

FIG. 2 shows a schematic cross-sectional view of a germicidal light emitting device according to another embodiment of the invention.

FIG. 3 shows a schematic cross-sectional view of a germicidal light emitting device according to yet another embodiment of the invention.

As illustrated in the figures, the sizes of layers and regions are exaggerated for illustrative purposes and, thus, are provided to illustrate the general structures of embodiments of the present invention. Like reference numerals refer to like elements throughout.

### DETAILED DESCRIPTION

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and fully convey the scope of the invention to the skilled person.

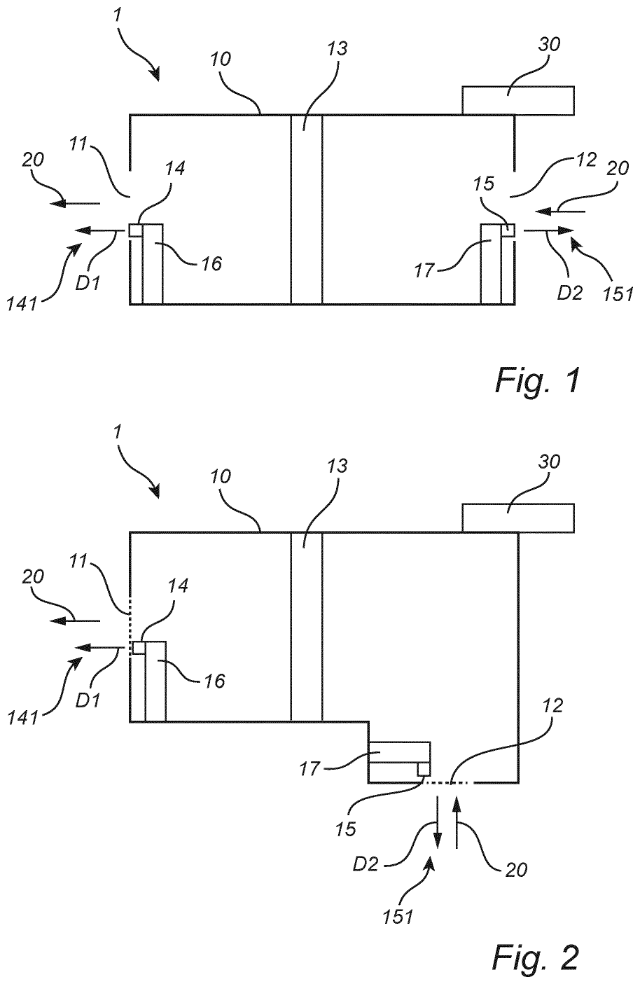

Referring initially to FIG. 1, a schematic cross-sectional view of a germicidal light emitting device 1 according to an embodiment of the invention is shown. The germicidal light emitting device 1 comprises a housing 10. The housing 10 may be a plastic or metallic housing. The housing 10 may be transparent or opaque. Preferably, the housing 10 is made from a material which does not degrade due to exposure of UV light, such as to provide for a more robust and durable light emitting device 1. The housing 10 comprises an air inlet 12 and an air outlet 11. The air inlet 12 and the air outlet 11 are formed as openings in the housing 10. Arranged within the housing 10 is a fan 13. The fan 13 is configured to provide an air flow 20 through the air inlet 12, through the housing 10 from the air inlet 12 to the air outlet 11 and through the air outlet 11. The fan 13 may be formed as a propeller with one or more blades rotating around a center axis and configured to provide the air flow 20 via rotation of the blades. The fan 13 may be arranged between the air inlet 12 and the air outlet 11, such as in a flow path or an air flow path between the air inlet 12 and the air outlet 11. The air flow 20 is directed through the air inlet 12 towards a first direction D1 and towards the air outlet 11 and away from a second direction D2. In the shown embodiment the first direction D1 is opposite the second direction D2. The air flow 20 may be a forced air flow 20. It is noted that in an embodiment the fan 13 may be omitted, and the air flow may be obtained by using a chimney effect. However, a forced fluid flow created by a fan provides a higher disinfection performance.

The germicidal light emitting device 1 further comprises a first UV light source 14. The first UV light source 14 is arranged and configured to emit, when in operation, first UV light 141 with a first spectral distribution and a main emission direction being the first direction D1. The germicidal light emitting device 1 further comprises a second UV light source 15. The second UV light source 15 is arranged and configured to emit, when in operation, second UV light 151 with a second spectral distribution and a main emission direction being the second direction D2. In the shown embodiment the first UV light source 14 and the second UV light source 15 are arranged within the housing 10. The first UV light source 14 is arranged to and configured for emitting light 141 out of the housing 10 through the air outlet 11. The second UV light source 15 is arranged to and configured for emitting light 151 out of the housing 10 through the air inlet 12. The first UV light 141 emitted by the first UV light source 14 is UV-C light. The second UV light 151 emitted by the second UV light source 15 is UV-B light. The first UV light source 14 and the second UV light source 15 are mounted on a first stand 16 and a second stand 17, respectively. The stands 16, 17 are optional elements. The stands 16, 17 are connected to an interior wall of the housing 10. In other embodiments, the stands 16, 17 may be connected to an exterior wall of the housing 10, and consequently, the first UV light source 14 and the second UV light source 15 are arranged outside the housing 10. The fan 13 may be arranged between the at least one first UV light source 14 and the at least one second UV light source 15.

The germicidal light emitting device 10 further comprises a mounting element 30. The mounting element 30 is attached to an exterior wall of the housing and allows the germicidal light emitting device 10 to be mounted onto a surface, such as but not limited to a wall or a ceiling or a post. The mounting element 30 as such is an optional element, but claimed germicidal light emitting devices relate only to any embodiment of a germicidal light emitting device 10 according to the invention comprising said mounting element. For example, and in particular in embodiments where the first direction D1 is perpendicular to the second direction D2, and where the first UV light 141 is UV-C light and the second UV light 151 is one or more of UV-B and UV-A light, i.e. is not UV-C light, the mounting element 30 may be configured to enable mounting the germicidal light emitting device on a surface in such a way that the first direction (D1) extends in parallel to the surface. In case the surface is a ceiling, such a germicidal light emitting device 10 enables upper air disinfection without exposing persons below the germicidal light emitting device 10 to the more dangerous UV-C light. It is noted that in some, not claimed embodiments, the first UV light 141 does not comprise UV-B and UV-A light. It is noted that in some, not claimed embodiments, the second UV light 151 does not comprise UV-C light.

The ultraviolet wavelength range is defined as light in a wavelength range from 100 to 380 nm and can be divided into different types of UV light/UV wavelength ranges. With reference to Table 1, different UV wavelengths of radiation may have different properties and thus may have different compatibility with human presence and may have different effects when used for disinfection. In Table 1, a + sign indicates that light in the specific range has the indicated effect, while a − sign indicates that the light in the specific range does not have the indicated effect. A+/− sign indicates a moderate effect. As mentioned below, the effect may in itself be desired or undesired (e.g. ozone generation).

TABLE 1
Properties of different types of UV wavelength light

| | | | (Relative) | | | | |
|---|---|---|---|---|---|---|---|
| | Short | Wavelength | sterilization effectiveness | | Safe | Vitamin D | Ozone |
| Name | name | (nm) | Bacteria | Viruses | Radiation | generation | generation |
| Violet | V | 380-420 | +/− | − | + | | |
| Ultra- | UV-A | 315-380 | + | − | + | | |
| violet A | | | | | | | |
| Ultra- | UV-B | 280-315 | + | +/− | +/− | + | |
| violet B | | | | | | | |
| Near | Near | 230-280 | + | + | − | | |
| ultra- | UV-C | | | | | | |
| violet C | | | | | | | |
| Far ultra- | Far UV | 190-230 | + | + | + | | +/− |
| violet | | | | | | | |
| Extreme | Extreme | 100-190 | + | + | − | | + |
| ultra- | UV-C | | | | | | |
| violet C | | | | | | | |

Each UV type/wavelength range may have different benefits and/or drawbacks. Relevant aspects may be (relative) sterilization effectiveness, safety (regarding radiation), and ozone production (as result of its radiation). Depending on an application a specific type of UV light or a specific combination of UV light types may be selected and provides superior performance over other types of UV light. UV-A may be (relatively) safe and may kill bacteria but may be less effective in killing viruses. UV-B may be (relatively) safe when a low dose (i.e. low exposure time and/or low intensity) is used, may kill bacteria, and may be moderately effective in killing viruses. UV-B may also have the additional benefit that it can be used effectively in the production of vitamin D in a skin of a person or animal. Near UV-C may be relatively unsafe but may effectively kill bacteria and viruses. Far UV may also be effective in killing bacteria and viruses but may be (relatively to other UV-C wavelength ranges) (rather) safe. Far-UV light may generate some ozone which may be harmful for human beings and animals. Extreme UV-C may also be effective in killing bacteria and viruses but may be relatively unsafe. Extreme UV-C may generate ozone which may be undesired when exposed to human beings or animals. In some application ozone may be desired and may contribute to disinfection, but then its shielding from humans and animals may be desired. Hence, in Table 1 “+” for ozone production especially implies that ozone is produced which may be useful for disinfection applications but may be harmful for humans/animals when they are exposed to it. Hence, in many applications this “+” may actually be undesired while in others, it may be desired.

Referring to FIG. 2, a schematic cross-sectional view of a germicidal light emitting device 1 according to another embodiment of the invention is shown. The germicidal light emitting device 1 shown in FIG. 2 differs from the one shown in FIG. 1 in that the first direction D1 is non-parallel with the second direction D2. In the shown embodiment, the first direction D1 is perpendicular to the second direction D2. Furthermore, the air inlet 12 and the air outlet 11 comprises a plurality of openings, slits or holes. The plurality of openings allows an air flow 20 to pass through the air inlet 12 and the air outlet 11.

The air outlet 11 may be configured to collimate the first UV light 141 along the first direction D1, i.e. such that the first UV light 141 propagates in parallel with the first direction D1. Additionally, or alternatively, the air inlet 12 may be configured to collimate the second UV light 151 along the second direction D2, i.e. such that the second UV light 151 propagates in parallel with the second direction D2. For instance, the plurality of openings may also be configured to collimate light 141, 151 emitted from the UV light sources 14, 15 arranged within the housing 10. In the shown embodiment the plurality of openings comprised by the air inlet 11 have a collimating effect on light 141 emitted by the first UV light source 14. The plurality of openings comprised by the air inlet 12 have a collimating effect on light 151 emitted by the second UV light source 15. The plurality of openings, slits or holes of at least one of the inlet 12 and the outlet 11 may be light absorbing such as to further collimate the light 141, 151.

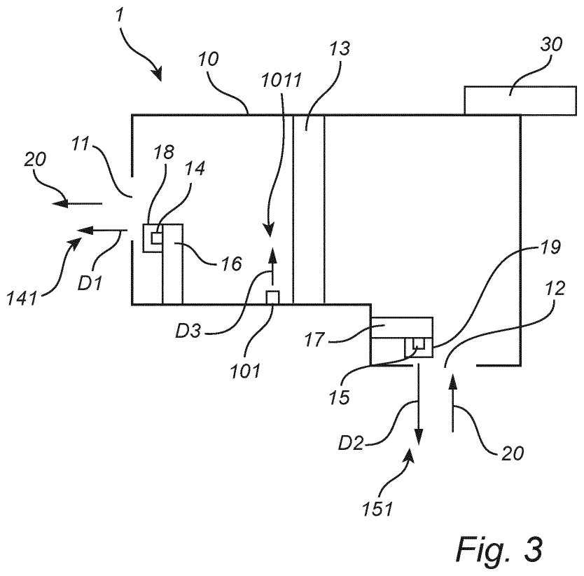

Referring to FIG. 3, a schematic cross-sectional view of a germicidal light emitting device 1 according to yet another embodiment of the invention is shown. The germicidal light emitting device 1 shown in FIG. 3 differs from the one shown in FIG. 2, in that the air inlet 12 and the air outlet 11 only comprises a single opening each. Furthermore, in the shown embodiment the germicidal light emitting device 1 further comprises a first collimator 18 and a second collimator 19. The first collimator 18 is arranged at the first UV light source 14 and configured to collimate the first UV light 141 along the first direction D1. The first collimator 18 may comprise any one or more of reflector, a total internal reflection collimator, or lamellae. The second collimator 19 is arranged at the second UV light source 19 and configured to collimate the second UV light 151 along the second direction D2. The second collimator 19 may comprise any one or more of reflector, a total internal reflection collimator and lamellae. The first collimator 18 and the second collimator 19 may be different or may be the same.

The germicidal light emitting device 1 further comprises a third UV light source 101. The third UV light source 101 is configured to emit, when in operation, third UV light 1011 with a third spectral distribution within the housing 10. The third spectral distribution is different from the first spectral distribution and the second spectral distribution, respectively. In the shown embodiment, the third UV light source 101 is configured to emit light along a third direction D3. Optionally, a third collimator may be arranged at the third UV light source 101 and configured to collimate the third UV light 1011 along, e.g., the third direction D3, i.e. such that the third UV light 1011 propagates in parallel with the third direction D3. The third collimator may comprise any one or more of reflector, a total internal reflection collimator and lamellae. The third collimator may be different from or may be the same as at least one of the first collimator 18 and the second collimator 19.

The person skilled in the art realizes that the present invention by no means is limited to the preferred embodiments described above. On the contrary, many modifications and variations are possible within the scope of the appended claims.

Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

1. A germicidal light emitting device comprising:
a housing forming an air inlet and an air outlet, configured to allow an air flow to pass in an air flow direction,
a fan configured to provide an air flow through the air inlet, from the air inlet to the air outlet and through the air outlet, the air flow being directed in a first direction to a second direction different from said first direction,
at least one first UV light source arranged and configured to emit, when in operation, first UV light with a first spectral distribution and an emission targeted in the first direction along or towards a ceiling/or a wall,
at least one second UV light source arranged and configured to emit, when in operation, second UV light with a second spectral distribution and an emission targeted in the second direction, and
at least one mounting element configured to enable mounting the germicidal light emitting device on the ceiling or the wall in such a way that the first direction extends in parallel to the ceiling or the wall,
wherein the first UV light is UV-C and the second UV light is at least one of UV-A and/or UV-B, and
wherein at least one of the first and second UV light source are arranged and configured for emitting light out of the housing.

2. A germicidal light emitting device according to claim 1, wherein both first and second UV light source are arranged and configured for emitting light out of the housing.

3. A germicidal light emitting device according to claim 1, wherein both first and second UV light are targeted along or towards the ceiling and/or a wall.

4. A germicidal light emitting device according to claim 1, wherein the air outlet is configured to collimate the first UV light along the first direction.

5. A germicidal light emitting device according to claim 1, wherein the air inlet is configured to collimate the second UV light along the second direction.

6. A germicidal light emitting device according to claim 1, wherein the first direction is any one of:
non-parallel with the second direction,
perpendicular to the second direction, and
opposite the second direction.

7. A germicidal light emitting device according to claim 1, further comprising at least one of a first collimator and a second collimator, wherein the first collimator is arranged at the at least one first UV light source and configured to collimate the first UV light along the first direction, wherein the second collimator is arranged at the at least one second UV light source and configured to collimate the second UV light along the second direction, wherein the first UV light comprises a first spatial light distribution having a first full-width-half-maximum, FWHM1, and the second UV light comprises a second spatial light distribution having a second full-width-half-maximum, FWHM2, wherein FWHM1<FWHM2, preferably FWHM1<0.5*FWHM2, more preferably FWHM1<0.3*FWHM2, and most preferably FWHM1<0.1*FWHM2.

8. A germicidal light emitting device according to claim 1, further comprising at least one third UV light source configured to emit, when in operation, third UV light with a third spectral distribution and a main emission direction being the third direction, wherein the third spectral distribution is different from the first spectral distribution and the second spectral distribution, wherein the third direction is different from said first and second direction.

9. A germicidal light emitting device according to claim 1, wherein the first direction is perpendicular to the second direction.

10. A germicidal light emitting device according to claim 8, wherein the third UV light is emitted in a third direction, wherein a second air flow is provided in a third direction, and wherein the third direction is perpendicular to the first direction.

11. A germicidal light emitting device according to claim 1, wherein at least one of the air inlet and the air outlet comprises a plurality of slits, holes, or openings.

12. A germicidal light emitting device according to claim 1, wherein at least one of the first UV light source and the second UV light source is arranged inside the housing.

13. A germicidal light emitting device according to claim 1, and further comprising a controller for individually controlling one or more of the first UV light source, the second UV light source and, where provided, the third UV light source.

14. A luminaire or a lamp comprising a germicidal light emitting device according to claim 1 and a light source emitting, when in operation, white light.

15. A luminaire or a lamp comprising a germicidal light emitting device according to claim 1, wherein the white light has a main emission direction being the second direction.
